(19) United States
(12) Patent Application Publication
(10) Pub. No.: US 2022/0125624 A1
(43) Pub. Date: Apr. 28, 2022

(54) Itch Blower

(71) Applicants: Huanchen Li, Westford, MA (US); Wendy Wang, Westford, MA (US)

(72) Inventors: Huanchen Li, Westford, MA (US); Wendy Wang, Westford, MA (US)

(21) Appl. No.: 17/509,150

(22) Filed: Oct. 25, 2021

Related U.S. Application Data
(63) Continuation-in-part of application No. 63/110,944, filed on Nov. 6, 2020.
Application No. 63/110,944 is a continuation-in-part of application No. 63/106,331, filed on Oct. 27, 2020.

(51) Int. Cl. A61F 7/00

(52) U.S. Cl. CPC A61F 7/007; A61F 2007/0086; A61F 2007/006; A61F 2007/0001; A61F 2007/0078

(57) ABSTRACT

The Itch Blower is hand held, have a way to determine the distance from the skin. It blows hot air with the right temperature for eliminating the skin itching. Most importantly, it can sense the skin temperature remotely and shut off the heating automatically so to avoid burning. This will guarantee a smooth approval from the FDA and a great success in commercialization. The device eliminates skin itching completely in just seconds, no matter how bad the itching is, and keeps the itching away for up to 24 hours, and when the itching comes back, patients can use the device to quickly blow away the itching again, so to stay itching free. It can also clear up acne and eczema for some patients.

### CROSS REFERENCE TO RELATED APPLICATIONS

This application is a continuation-in-part of the provisional application Ser. No. 63/106331 filed on Oct. 27, 2020 and provisional application Ser. No. 63/110944 filed on Nov. 6, 2020

### BACK GROUND OF THE INVENTION

The FDA put safety first; they will not approve your device if it has a safety concern. Companies also want safety; only a safe product can guarantee a successful commercialization. Using heat to eliminate skin itching has been very successful with our earlier product Electronic Itch Stopper (https://www.ItchStopper.com). The heat must be high that it is at the edge of causing skin burning. So it is not safe if without a strict control.

If the heat is not scalding, it will not work on Itching. Why people have not found that a hair dryer work on skin itching? One reason is that a hair dryer will work on itching only if you put it so close to the skin that it causes a scalding sensation. People will quickly move the hair dryer away from the skin when they got those sensations. However, to clear up the itching, you need to continue the heating for 1-7 seconds with that scalding sensation.

So far, nothing works on large areas of skin itching like those caused by eczema, poison ivy/oak, herpes, hives, allergy, cold sore, cancer, renal or liver failures, etc. Cortisone creams can work to a certain degree, but they don't stop the itching instantly, and you cannot use them for more than 14 days or it will cause permanent damages to your adrenal glands. You can find such warnings on those anti-itching products, and you need a stop for about one month before using them again.

Many people got poison ivy/oak. The itching was unbearable, and lasted for about three months. It was a horrible experience for the sufferer. As a medical doctor, I also know many patients simply scratched their skin to bleeding. About 50% of the population has herpes, and some of them also have itching that is unbearable. In the U.S., about 10% people badly need professional medical help for their skin itching.

Twenty years ago, the FDA approved our medical device, Electronic Itch Stopper (https://www.ItchStopper.com), we have sold a lot of it and saved countless of skin itching patients. Some patients said that the Itch Stopper saved their lives and others said they were totally dependent on it. We are proud for what we have done.

Fifteen years ago, we sold our patents about the Itch Stopper to a U.S. company. That company got a FDA approval for clearing up the acne. Although the Itch Stopper works on acne, its success rate is only about 70%, this cannot guarantee a big commercial success because of a high return rate. However, for skin itching, it's a sure thing. The success rate is 100% for skin itching.

The Itch Stopper has some limitations. It uses a hot plate to heat the skin, can only treat very localized skin itching. It does not work on large areas of skin itching, yet, only such large areas of skin itching wanting treatments most. The other problem associated with the Itch Stopper is it works only on flat, but not curved, skin surface. A third problem is that it is not good for blistering skin, yet most sever patients having the blistering. Many patients are asking us to make larger and curved Itch Stopper. To satisfy such an unmet need, we invented the Itch Blower for the treatment of skin itching and most skin diseases.

The Itch Blower uses hot air as the medium to deliver the heating energy. It's very similar to a hair dryer but will have some modifications. Our modifications will make a hair-dryer safe and effective for the treatment of skin itching. The Itch Blower can cover large areas, can be used on curved areas, and can be used on blistering skin. It will work for the treatment of whole body skin itching. The Itch Blower is the answer for all kinds of skin itching.

Itch Blower VS Hair Dryer, while you will have a few occasions when you can swap one out of the other, a hair dryer will mostly fail on skin itching. Firstly, when you put the hair dryer too close to the skin, it causes injuries. Secondly, when you put it to the working distance for itching, you feel a scalding, and most people will simply move it away because of the scalding. They simply will not heat the skin for an enough time that cures the itching. Thirdly, when you put the hair dryer too far away from the skin, it worsens the itching because it simply warm up the skin. Warming up the skin without to the degree of scalding will cause an increase in the local blood flowing which worsens the inflammation; it will also cause the skin Mast cells release more histamine which worsens the itching. This is why people feel much more itching when they get hotter.

An antigen, like one injected by a mosquito, will first bind with two antibodies to form a trimmer which triggers a flushing of calcium from the calcium channels, and then, causes the skin mast cells degranulate the histamine which causes the itching, rash, skin bumping, inflammation and most skin diseases such as acne. Warming up the skin will simply accelerate such a process so to cause more itching. However, scalding the skin will not. It will stop the process. It will cause the antigen to bind with more than two antibodies so to form a multi-mer that does not trigger the calcium flush, according to a report; also, scalding the skin may denature some calcium channel degranulating-proteins, so to stop the itching.

What we want is to scalding the skin without burning it.

Our skin is well equipped in handle the scalding. We will feel stinging, tingling, scalding, slight pain, or slight burning sensations in this range, and when well controlled to stay at such sensations for a short time, we can kill the itching. This effective temperature rang is very narrow. Without ways of controlling, we will either worsen the itching or burning the skin. Before the burning happens, those feelings will become unbearable, and, if we control the heating temperature high enough to kill the itching but low enough to give us time to withdraw from the heating source, we will be safe.

### EMBODIMENTS

The Itch Blower may comprise all the elements a haft dryer does. However, we will remove all elements that may affect it from working on skin itching. And furthermore, our itch Blower uses hot air for heating the skin rather than the hairs. We will modify a hair-dryer by setting the hot air to certain temperatures so that it heats the skin to certain working temperatures for specific itching cases at certain air flow rate and at certain distance between the skin and the air opening. This means, with a specific air flow rate and a specific distance, we will chose a heating wire capacity that can keep the hot air so hot as to heat the skin to a effective temperature, such as 43° C. or 52° C., quickly, and for a specific duration such as 3 seconds, so that it can stop the skin itching, but without causing burning.

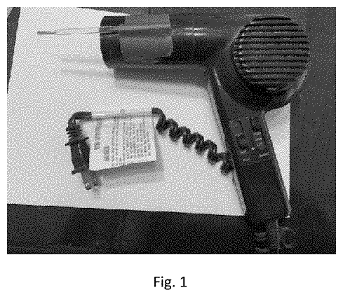

To let users know how far away they must put the Itch-Blower-hot-air-opening from the skin, we can simple put a label, such as “1 inch”, on the device or in the users manual; we may also attach a stick (FIG. 1), which means a stick may protrude from the opening of the Itch Blower toward the skin. One end of the stick is attached to the opening, and the other end of the stick points to the skin. When the users put the stick on or near the skin, the users know the desired distance. The length of the stick can be adjustable, which means it can be pull longer or pushed shorter. We may put labels, such as child, adult leg, adult stomach, thicker than normal skin, and etc, at each different length, so that when the users adjust the length, they can see the labels to tell whether that is for their cases of itching.

Replacing the physical stick, we can incorporate an infrared, laser or any other distance measurers into the Itch Blower, for measuring the distance remotely, and, in this cases, we use a digital signaling means, such as LED screen, to tell the user to put the Itch Blower closer to the skin, or further away from the skin, or it's already the right distance.

Other than a LED screen, we can use any other ways for showing the distance settings, and for informing users whether the distance is right.

See FIG. 1, the stick can be a thermometer, with a second function as to sense the temperature of the hot air near the skin or directly sensing the temperature of the skin, When the hot air or the skin reaches the desired temperature, and for a desired time, the sensor may work with a thermostat to lower the heating energy so to control or lower down the temperature of the hot air, lower down the volume of the hot air so to let the skin keep at a safe but working temperature, or even shut off the device intermittently so to avoid the burning of the skin. Alternatively, mercury can be put inside the stick in a same way as people do with a thermometer, with the tip containing more mercury. The temperature causes the mercury to expend, when the mercury goes to a certain length, it turns off the power in the way similar to what we described in our earner patent (FIG. 3 in Patent number: U.S. Pat. No. 7,537,605 B2)

A thermo couple can do a better job in sensing the temperature and feeding back to the thermostat, infrared and any other non contact and remote temperature sensors can do a better job too. We should incorporate these into our Itch Blower. For the purpose of how to use a thermocouple or a digital temperature sensor, a technician may refer to stove ovens, home heating system thermostats, or any other machines, such as a infrared body temperature sensor.

We need to incorporate a temperature selector so that people can set their desired type of treatments. The temperature selector can work with a thermostat which, in turn, can work with the temperature sensor and, may also work with the distance measurer. Software may be developed so to make the thermostat smart, like a robotic thermostat.

Different cases of skin itching may need different temperatures.

Such as with our earlier itch Stopper, a heating pad is about 49° C. for treating children skin itching, about 51° C. for adult body skin itching, about 53° C. for adult limb skin itching, and higher temperatures, such as 57° C., for chronic skin itching patients who's skin has getting thicker than normal, and 62° C., for even worse cases. For example, when a patient set the setting to Setting 1 or adult-leg, the Heating pad may have a temperature of around 53° C.

The Itch Blower may do the same, to blow different temperatures for different treatments. Since the itch Blower does not contact the skin, the temperatures may be a little higher, such as 2° C. higher than the itch Stopper does. We will do more detailed study to get more accurate temperatures for each specific treatments with the hot air.

Skin temperatures are different from the above heating pad or hot air temperatures, The Itch Blower can sense the skin temperature directly; we can have it heat the skin to specific temperatures. We will do much more extensive tests, but currently, we intend to heat the skin to a temperature about 2-5° C. lower than the temperatures our earlier Itch Stopper offers for each specific treatment.

The temperature of the skin has a working range, In the above, we said our heating pad offers 53° C. for adult limb skin itching, but when come to the skin; the working range can be 47-53° C., or even lower.

If we incorporate only a temperature sensor, either remote sensor or contact sensor, for sensing the skin temperature, the distance from the opening to the skin becomes less important. So in this case, we may not need to incorporate the distance sensor. We may simply recommend the users to put the Itch Blower to about 1 inch or any other distances. We may simply use a normal hair dryer, and let the temperature sensor to tell the thermostat the skin temperature. When the skin temperature reaches a specific case that is set by the user, the thermostat may lower or shut off the power of the heating element and/or the fen, so to lower the temperature of the hot air or even stop the heating, When the skin temperature get lower than the desired temperature, the thermostat may turn back the power, this way, intermittently, so to keep the skin temperature in the working range.

While we prefer the skin temperature stays within 42-60° C., but the hot air temperature can be much higher. Any hot air temperature between 42 to 1000° C. may be used. The duration for a single treatment for heating a skin area can be 2-7 seconds in a single treatment, although any other duration such as 1-150 seconds can be used.

The hot air temperature becomes less important when we can measure the skin temperature; we can use a high hot air temperature, Hire dryer type Y260SR, model 259Y, 125V AC, 60 Hz, 1875 W, which is manufactured by Conair Corporation has three settings, for controlling the temperature of the hot air at three different temperature levels. We may remove these settings and keep the hot air temperature to only high level, then incorporate an IR remote temperature sensor (remote thermometer). We may add three settings for controlling the skin temperature. When the thermometer sees the skin temperature reaches a desired temperature (working range for a specific treatment), it will tell the thermostat to lower or shut off the power of the heating elements so to lower the temperature of the hot air or even shut off the fen so to stop the heating. Such as, when the thermometer see the skin temperature reaches a desired temperature for children, it will tell the thermostat to lower or shut off the power to the heating wire or the fen so to lower or stop the heating. When the thermometer see the skin temperature reaches a desired temperature for adult legs, the Itch Blower will do the same.

When designing the Itch Blower, a technician may refer to a thermostat that works in our home digital heating system or digital remote body thermometer. it should clearly show how a temperature sensor tells the thermostat to shut off and turn on the heating system, and in our case, how the thermostat shut off and turn on our heating element or the fen when the skin temperature reaches the desired setting. In case we just want to lower down the strength of the heating elements, we may do that in the same way but just lower down the power supply to the heating elements by switching the power to an electric resistor with stronger Ohms, so that less electricity can go through the heating elements. Vise versa.

The hot air that comes out from a normal hair dryer is not evenly heated or not well mixed. It is hotter around because of the heating wires are around, but not that hot in the center because the center does not have hot wire. What we will do is to adding some heating elements to the center, such as mess up the wire so every where including the center will have the heating wire. We may also change the hot air pass-way, such as making one or few turns, so to let the air get mixed before coming out.

The opening of a normal hair dryer is too wide for some localized itching, such as a mosquito bit. We want the opening to be adjustable, so that, the users can adjust the size of the opening according to the size of the itching. They can use the biggest opening to treat very large areas so that they can cover even the whole body, relatively smaller opening to treat a large area of localized itching, and the smallest opening for a localized itching like a insect bit.

A simplest way is to attach a piece of non-combustible cloth sleeve, like a shirt sleeve, to the opening. One end of end of the sleeve is attached to the opening the Itch Blower and the other end is free and open. The free end of the sleeve-opening may have a rounded rope like a waist belt, people can adjust the opening of the sleeve by tightening or relaxing the rounded rope. This will allow users to control the width of the air so to treat differently sized itching.

We can also make sized openings. One end of the sized-opening can be easily attached to the opening of the Itch Blower, and the other end is sized. When users want to treat a large area itching, they attach a big opening. When come to a small area itching, they attach smaller opening.

All other technologies in other fields may be employed. Such as with a traditional camera, you can adjust the size or the focus by simply turning a controller at the tip, and the many different pieces of metal-plates that are attached at the opening can overlap more or less so to narrow or widen the opening.

Either batteries or a house main power supply can be used as the power source.

In another embodiment, we can simply put a label on the device or a notice in the user's manual. The label or the notice will ask the patient to put the Itch Blower close enough, like only ½ inch to 1 inch, to the skin so that the Itch Blower can heat the skin to the effective temperatures. The label/notice may also tell the patients that they must continue heat the skin for a duration, such as 1-7 seconds, when they have already felt a kind of stinging, tingling, slight pain, scalding, and/or slight burning sensation.

A more advanced Itch Blower, which incorporates a laser distance sensor to tell the distance, an infrared remote temperature sensor to tell the skin temperature, and a robotic thermostat. After the user selected a setting, such as for adult leg, the robotic thermostat will set the hot air temperature and air flow rate according to the distance, so to quickly heat the skin to the desired temperature, and once the skin reaches the desired temperature, the robotic thermostat can let the skin stay at the desired temperature by lowering down the hot air temperature through lowering down the supply of power to the heating element, or by slowing down the hot air blowing rate through slowing down the fen, or by turning off and on the Itch Blower intermittently. The temperature of the air or the flow rate of the air can be constant. It can also be constantly changing, such as it goes up and down, so to keep the skin temperature at the effective temperature for the skin itching.

We may also develop and incorporate software that can tell that a dose of heating energy has been delivered to the skin for eliminate an Itching, and can tell the thermostat to shut off the Itch Blower when it happens.

To let a remote distance measurer work with a thermostat, we can refer to the ways that are employed by our home heating systems. When the measurer tells the thermostat the distance is far, the thermostat will switch an electric wire to go through a resistor with smaller Ohms, so the heating elements and/or the fen can get more power. This ways, a higher temperature and higher blow rate of the hot air will blow out to the skin. Vise versa.

The working distance between the device-opening from the skin is variable. It depends on the hot air temperature at the opening and the air flow rate. With a normal hair dryer that has a temperature of around 150 F, the distance can be around 1 inch. However, any other distance from inches ¼ to 2 feet may be used as long as the Itch Blower can heat the skin surface to around the desired temperature for a specific case. The thermostat can get the distance from the distance measurer. When we want to determine what distance is good for a specific case, we need to fix the hot air temperature and the air flow rate at the device opening. We may simply use the hot air temperature and air flow rate of a hair dryer that we purchase from the market. Then we measure the temperature of the hot air at around ½ to 3 inches from the opening with a separate temperature sensor. In order to heat the skin to a desired temperature, the hot air temperature must be 2 or more degrees higher, So that if the temperature at that distance is around 51° C., we know that distance is good for heating the skin to around 49° C. for children skin itching, and we can design the thermostat to set the hot air temperature and/or the air flow rate to that strength, and mark that distance for child use. If it is around 52° C., we know that is for heating the skin to 50° C. for adult body skin itching, and do the same as the above, and so on. We may recommend those distances to users in the user's manuals, or put signs on the adjustable stick at those distances. In case we incorporate a remote distance measurer, we may use a LD screen to show the users that they are using a distance for a recommended case.

We may design an Itch Blower that incorporates both a temperature sensor and a distance measurer. We can design an Itch Blower that provide higher hot air temperature and/or higher air blow rate when a user put the distance far, and verse vise. Then let the temperature sensor to measure the skin temperature and tell the thermostat to turn on or off the heating element and/or the air fen.

To make sure the Itch Blower always work on itching, we may add an automatic switch that turns off the power of the fen and/or the heating element whenever the temperature sensor feels that distance is too far away from the skin, and turn on them automatically only when the distance is in the effective range.

To make the sure the Itch Blower is safe, the automatic switch can also automatically turn off the device when the distance is too close to the skin, like only ¼ inches, as to avoid causing injuries to the skin. It will also turn off the Itch Blower when the skin temperature reaches an desired temperature for a specific treatment or an unsafe temperature, like 62° C. for an adult leg.

The automatic switch will turn back on the power after the device get within the desired distance from the sink, after a short time such as one second, or after the skin cools down.

Heating can be dosed, when the skin reaches a working temperature for a specific case like a adult leg, and stayed there for a specific time, like about 7 seconds for a adult leg, the device may shut off the heat for a time such as 1-3 second then restart.

Method embodiments should include steps like turning on a device that can blow hot air, aiming the hot air to a skin area that has itching, moving the device to the skin area to a distance that works for skin itching, and allowing the hot air to blow to the skin area for the purpose of clearing up the skin-itching, and remove the device when you feel a scalding feeling for a few seconds.

The device must be positioned from the skin area with a distance that works for the skin itching. The hot air temperature at the opening and the hot air blowing rate will determine the distance. For example, if we design an Itch Blower that is similar to the Conair hair dryer type Y2605R, model 259Y, 125V AC, 60 Hz, 1875 W, we may further confine the temperature ranges at different settings. When an Itch Blower is designed to blow hot air of about 120° F. at the opening and at the air flow rate similar to that of the above Conair hair dryer at high setting, the user should position the opening of the Itch Blower to the skin surface at around ½ inch. When an Itch Blower is designed to blow hot air of about 150° F. at the opening, the user should position the opening of the device to the skin surface at around 1 inch. When an Itch Blower is designed to blow hot air of about 180° F., the user should position the opening of the device to the skin surface at 1-2 inches. When an itch Blower is designed to blow hot air of about 200° F., the user should position the device to the skin surface to around 2-3 inches. As mentioned about, we may put all these information in the user's manual or on the surface of the Itch Blower.

The method should also have a step for allowing the hot air to continue blowing to the skin area until the user feels a stinging, tingling, scalding, slight burning or slightly painful sensations for a few seconds.

1. A system comprising a plurality of apparatus, each comprising:
a heating means for heating air, a fen means for blowing the air through said heating means to the skin, a thermostatic means for controlling the hot air temperature, a power source means for provide enough power to every means;
an user's manual or a label for instructing a user to use the apparatus for treating a skin disease such as a skin itching, to put the apparatus to a specific working distance, such as 1 inch, from the skin, and/or to forcefully continue the treatment for a specific time, such as 1-7 seconds, when the user already feels a tingling, scalding, slight burning, or slight painful sensation before stopping the treatment.

2. The system as in claim 1, where in, each apparatus further comprising a temperature sensor for directly determining the skin temperature.

3. The system as in claim 1, where in each apparatus further comprising a distance means, such as a stick or a laser distance measurer, for helping the users to put the apparatus to a working distance for skin disease.

4. The system as in claim 2, where in, each apparatus further comprising an automatic switch that works like a thermostat, for shutting off or lowering down the power of the heating means so to lower down the temperature of the hot air or even shutting off the fen means so to stop the heating, when the temperature sensor tells it a desired temperature of the skin or the air for a specific treatment is reached, and/or an unsafe skin temperature is reached, and then, automatically turn back the power after the skin or the hot air cools, or simply turn back the power after a short time such as one second.

5. The system as in claim 4, where in each apparatus further comprising a distance means, such as a stick or a laser distance measurer, for helping the users to put the apparatus to a working distance for skin disease.

6. The system as in claim 5, where in each apparatus further comprising a software means for correlating all necessary means so that the thermostat can set the hot air temperature according to the distance.

7. The system as in claim 1, where in each apparatus further comprising a means for determining that a right dose of heat has been delivered and shut off the apparatus once done.

8. The system as in claim 1, where in each apparatus further comprising a means for letting the hot air mix well with the cooler air before the air get out from the opening.

9. The system as in claim 1, where in, each apparatus further comprising a temperature sensor for directly determining the temperature of the hot air,

10. A method for the treatment of skin-itching comprising:
turning on a device that can blow hot air,
aiming the hot air to a skin area that has skin itching, moving and positioning said apparatus to said skin area to a specific distance as specified in the instruction that works for said skin itching, and allowing the hot air to blow to said skin area for the purpose of clearing up said skin-itching;

11. A method as in claim 10 further comprising:
forcefully allowing said hot air to continue blowing to said skin area for a specific duration after said user feels a stinging, tingling, scalding, slight burning, and/or slightly painful sensation, and, then, moving said device away from said skin area.
